(19) United States
(12) Patent Application Publication
(10) Pub. No.: US 2020/0382286 A1
(43) Pub. Date: Dec. 3, 2020

(54) SYSTEM AND METHOD FOR SMART, SECURE, ENERGY-EFFICIENT IOT SENSORS

(71) Applicant: The Trustees of Princeton University, Princeton, NJ (US)

(72) Inventors: Ayten Ozge Akamandor, Plainsboro, NJ (US); Hongxu Yin, Princeton, NJ (US); Niraj Jha, Princeton, NJ (US)

(21) Appl. No.: 16/961,415

(22) PCT Filed: Jan. 10, 2019
(86) PCT No.: PCT/US2019/012987

§ 371 Date: Jul. 10, 2020

Related U.S. Application Data
(60) Provisional application No. 62/615,475, filed on Jan. 10, 2018.

(51) Int. Cl. H04L 9/06; G16H 40/67; G06N 20/00; G16Y 40/50; G16Y 10/75; H03M 7/30

(52) U.S. Cl. CPC H04L 9/0643; G16H 40/67; H03M 7/3062; G16Y 40/50; G16Y 10/75; G06N 20/00

(57) ABSTRACT

According to various embodiments, an Internet of Things (IoT) sensor architecture is disclosed. The architecture includes one or more IoT sensor components configured to capture data and one or more processors configured to analyze the captured data. The processors include a data compression module configured to convert received data into compressed data, a machine learning module configured to extract features from the received data and classify the extracted features, and an encryption/hashing module configured to encrypt and ensure integrity of resulting data from the machine learning module or the received data.

### CROSS-REFERENCE TO RELATED APPLICATIONS

This application claims priority to provisional application 62/615,475, filed Jan. 10, 2018, which is herein incorporated by reference in its entirety.

### STATEMENT REGARDING FEDERALLY SPONSORED RESEARCH OR DEVELOPMENT

This invention was made with government support under Grant #CNS-1617628 awarded by the National Science Foundation. The government has certain rights in the invention.

### FIELD OF THE INVENTION

The present invention relates generally to Internet-of-Things (IoT) systems and, more particularly, to an IoT sensor node configured to employ signal compression and machine learning inference.

### BACKGROUND OF THE INVENTION

Technological advancements have led to a proliferation of Internet-of-Things (IoT) targeted at various applications. IoT range from a small sensor that reports on the health information of the user to an array of sensors and devices that cover a whole city to regulate traffic, ensure security, and monitor weather. The total number of IoT devices is expected to reach 80 billion by 2025 and generate 180 zettabytes (ZB), or 180×10²¹ B, of data, in total. Thus, ensuring data security and reducing energy consumption required for signal processing and data transmission are prominent challenges faced by IoT designers.

The first step in an IoT application is to collect data through IoT sensors. These sensors generate raw data that need to be processed before any action can be taken. Typically, the collected data is transmitted to a base station for processing. However, base stations often have limited processing and storage resources. In such cases, they can only carry out simple operations on the data, such as reformatting, compression/expansion, aggregation, etc. Following these operations, data is transmitted to cloud servers for further processing and decision making (i.e., to distill intelligence and, hence, impart smartness to the system). Although cloud servers have the required computational resources for signal processing and information extraction, data transmission from IoT sensors to cloud servers creates serious design challenges, such as security concerns, insufficient energy, and limited bandwidth.

To get around these obstacles, previous studies have suggested pushing data processing towards the edge of the IoT devices and implementing cryptographic techniques (i.e., encryption and hashing) on the collected data. However, although edge-side computing enables decision-making without the use of cloud resources and encryption and hashing strengthen security, the overall computational cost, and hence energy consumption, increases significantly.

As such, there is a need for IoT systems that simultaneously achieve smartness, security, and energy efficiency.

### SUMMARY OF THE INVENTION

According to various embodiments, an Internet of Things (IoT) sensor architecture is disclosed. The architecture includes one or more IoT sensor components configured to capture data and one or more processors configured to analyze the captured data. The processors include a data compression module configured to convert received data into compressed data, a machine learning module configured to extract features from the received data and classify the extracted features, and an encryption/hashing module configured to encrypt and ensure integrity of resulting data from the machine learning module or the received data.

According to various embodiments, a method for processing captured data on an Internet of Things (IoT) sensor architecture is disclosed. The method includes capturing data via one or more IoT sensor components and analyzing the captured data via one or more processors. The analysis includes compressing received data via a data compression module, extracting features from the received data via a feature extraction module, classifying the extracted features via a classification module, and encrypting and ensuring integrity of resulting data from the machine learning module or the received data via an encryption/hashing module.

According to various embodiments, a non-transitory computer-readable medium having stored thereon a computer program for execution by a processor configured to perform a method for processing captured data on an Internet of Things (IoT) sensor architecture is disclosed. The method includes capturing data via one or more IoT sensor components and analyzing the captured data via one or more processors. The analysis includes compressing received data via a data compression module, extracting features from the received data via a feature extraction module, classifying the extracted features via a classification module, and encrypting and ensuring integrity of resulting data from the machine learning module or the received data via an encryption/hashing module.

Various other features and advantages will be made apparent from the following detailed description and the drawings.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only exemplary embodiments of the invention and are not, therefore, to be considered to be limiting its scope, the invention will be described and explained with additional specificity and detail through the use of the accompanying drawings, in which:

FIG. 1 is a diagram of basic components of an IoT sensor according to an embodiment of the present invention;

FIG. 2A is a diagram of an alert notification scenario for IoT sensor data according to an embodiment of the present invention;

FIG. 2B is a diagram of a continuous notification scenario of IoT sensor data according to an embodiment of the present invention;

FIG. 3 is a table of IoT sensors and corresponding application areas according to an embodiment of the present invention;

FIG. 4A is a diagram of a sense-and-transmit IoT sensor architecture according to an embodiment of the present invention;

FIG. 4B is a diagram of a smart, secure, and energy-efficient IoT sensor architecture according to an embodiment of the present invention;

FIG. 5 is a diagram of a sense-and-transmit approach according to an embodiment of the present invention;

FIG. 6 is a diagram of a sense-compress-transmit approach according to an embodiment of the present invention;

FIG. 7A is a diagram of IoT sensor architecture employing direct computations on compressively-sensed data without classification according to an embodiment of the present invention;

FIG. 7B is a diagram of IoT sensor architecture employing direct computations on compressively-sensed data with classification for alert notification according to an embodiment of the present invention;

FIG. 7C is a diagram of IoT sensor architecture employing direct computations on compressively-sensed data with classification for continuous notification according to an embodiment of the present invention;

FIG. 8A is a diagram of IoT sensor architecture based on signal processing with direct transmission according to an embodiment of the present invention;

FIG. 8B is a diagram of IoT sensor architecture based on signal processing machine learning inference for alert notification according to an embodiment of the present invention;

FIG. 8C is a diagram of IoT sensor architecture based on signal processing with machine learning inference for continuous notification according to an embodiment of the present invention;

FIG. 8D is a diagram of IoT sensor architecture based on signal processing with compression according to an embodiment of the present invention;

FIG. 8E is a diagram of IoT sensor architecture based on signal processing with compression and machine learning inference for alert notification according to an embodiment of the present invention;

FIG. 8F is a diagram of IoT sensor architecture based on signal processing with compression and machine learning inference for continuous notification according to an embodiment of the present invention;

FIG. 9 is a diagram of IoT sensor applications for alert and continuous notification according to an embodiment of the present invention;

FIG. 10 is a table of supraventricular and ventricular ectopic beat detection performance according to an embodiment of the present invention;

FIG. 11 is a graph of total energy consumption of S-beat and V-beat detecting architectural paths according to an embodiment of the present invention;

FIG. 12 is a table of Parkinson's disease freezing of gait detection performance according to an embodiment of the present invention;

FIG. 13 is a graph of total energy consumption for Parkinson's disease freezing of gait detecting architectural paths according to an embodiment of the present invention;

FIG. 14 is a graph of total energy consumption for EEG seizure detection for various architectural paths according to an embodiment of the present invention;

FIG. 15 is a graph of compressed-domain classification for 19-class daily activity classification task accuracy according to an embodiment of the present invention;

FIG. 16 is a table of energy breakdown for 19-class human activity classification without compression according to an embodiment of the present invention;

FIG. 17 is a graph of total energy consumption for neural prothesis for various architectural paths according to an embodiment of the present invention;

FIG. 18 is a graph of an architectural path containing nonlinear transformation according to an embodiment of the present invention; and

FIG. 19 is a table of energy breakdown for 6-class chemical gas classification in a no-compression case according to an embodiment of the present invention.

### DETAILED DESCRIPTION OF THE INVENTION

The proliferation of Internet-of-Things (IoT) has led to the generation of zettabytes of sensitive data each year. The generated data is usually raw, requiring cloud resources for processing and decision-making operations to extract valuable information (i.e., distill smartness). Use of cloud resources raises serious design issues such as limited bandwidth, insufficient energy, and security concerns. Edge-side computing and cryptographic techniques have been proposed to get around these problems. However, as a result of increased computational load and energy consumption, it is difficult to simultaneously achieve smartness, security, and energy efficiency.

As such, generally disclosed herein is a novel approach to employ signal compression and machine learning inference on the IoT sensor node. An important sensor operation scenario is for the sensor to transmit data to the base station immediately when an event of interest occurs (e.g. arrhythmia is detected by a smart electrocardiogram sensor or seizure is detected by a smart electroencephalogram sensor) and transmit data on a less urgent basis otherwise. Since on-sensor compression and inference drastically reduce the amount of data that need to be transmitted, the result is a dramatic energy bonus relative to the traditional sense-and-transmit IoT sensor. A part of this energy bonus is used to carry out encryption and hashing to ensure data confidentiality and integrity. The effectiveness of this approach is analyzed on six different IoT applications with two data transmission scenarios: alert notification and continuous notification. The experimental results indicate that relative to the traditional sense-and-transmit sensor, IoT sensor energy is reduced by 57.1× for electrocardiogram (ECG) sensor based arrhythmia detection, 379.8× for freezing of gait detection in the context of Parkinson's disease, 139.7× for electroencephalogram (EEG) sensor based seizure detection, 216.6× for human activity classification, 162.8× for neural prosthesis spike sorting, and 912.6× for chemical gas classification. Thus, the disclosed approach not only enables the IoT system to push signal processing and decision-making to the extreme of the edge-side (i.e., the sensor node), but also solves data security and energy efficiency problems simultaneously.

In the embodiments generally disclosed herein, compressed-domain inference is used based on concepts such as compressive sensing and compressed signal processing (CSP). Depending on the inference outcome, the IoT sensor transmits the data or provides an alert whenever necessary. Since compressed-domain inference significantly reduces the amount of data that needs to be transmitted, a large energy bonus is obtained. This enables the sensor to also carry out encryption and hashing to ensure data confidentiality and integrity.

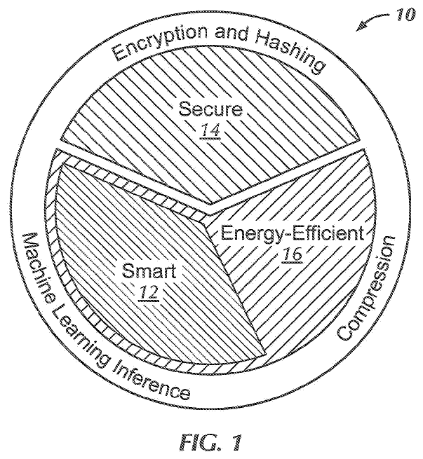

FIG. 1 shows the basic components of an IoT sensor architecture 10 according to an embodiment of the present invention. It achieves smartness 12 through decision-making inferences, security 14 through encryption and hashing, and energy efficiency 16 through both compression and decision-making inferences. The architecture 10 utilizes compression and decision-making inference to obtain a significant energy bonus relative to a traditional sense-and-transmit IoT sensor and uses a part of this bonus to also incorporate encryption/hashing on the sensor. The architecture 10 can be operated in multiple modes that easily adapt to various design objectives.

Motivation

IoT sensors are widely used in various applications such as healthcare, agriculture, industry, transportation, independent living, energy management and optimization, public safety, etc. Based on the user's needs and system goals, IoT applications either utilize a single sensor or an array of sensors included in a sensor node.

IoT sensors collect data from the environment. However, the collected data is typically raw (i.e., the data requires signal processing before any action can be taken) and the sensor node has limited processing and storage resources. In order to carry out the processing and make a decision, data is transmitted to cloud servers through base stations. However, data transmission poses serious design obstacles: insufficient energy, limited bandwidth, and security vulnerabilities. Limited available energy necessitates frequent battery replacement or recharging of the sensor node. This negatively impacts the practicality of the deployed IoT system. Limited bandwidth increases decision-making latency owing to busy Internet Protocol (IP) traffic. The IP traffic increases as the number of IoT devices connected to and utilizing the Internet increases, thus further exacerbating the bandwidth problem. CISCO's analysis of IP traffic provides support for this trend. This analysis indicates that the rate of IP traffic in 2016 was 1.2 ZB per year. In 2021, it is expected to increase by approximately 3 times, reaching 3.3 ZB per year.

In terms of security, various IoT sensors have been shown to be vulnerable to attacks that reveal sensitive information of the user or system. In one case, 73011 security cameras in a large number of countries were attacked. Since these cameras used publicly known login information (i.e., username and password), user privacy could be compromised without much effort. Moreover, recently a smart TV company was charged with collecting, storing, and marketing users' sensitive information without obtaining prior consent. In October 2016, a distributed denial-of-service (DDoS) attack was carried out in the United States and Europe by infecting a significant number of IoT devices with the Mirai malware. This is reported to be the most widespread DDoS attack in history. It exposed the security vulnerabilities of IoT devices and the seriousness of the consequences of deploying such devices. As another example, 70 on-market IoT sensors were analyzed from a security point of view. They demonstrated attacks on eight of these sensors when employed in two different IoT applications: residential and industrial automation/monitoring. In the case of the residential automation/monitoring system, they targeted motion, door, and smoke detector sensors. They could obtain the pin numbers of these sensors in a few seconds and reverse-engineer the wireless packet format of the sensors. In the industrial system setting, they targeted fluid level sensors. Reverse engineering of the packet allowed them to generate malicious packets that are not recognized as malicious by the base station. This allowed them to generate continuous false alarms in order to wear the user down into deactivating the alarm system, which could be a precursor to a more serious attack on the system.

To minimize these security vulnerabilities in IoT sensors, time-stamps, cryptographic techniques, an uncommon username/password combination, and a longer sensor identification number could be utilized. However, incorporation of these techniques adversely impacts the energy consumption of the IoT sensor by either increasing the packet size in data transmission or the amount of computation in processing and decision-making operations. For example, incorporating cryptographic techniques (i.e., encryption and hashing) increases energy consumption by approximately 150% compared to the traditional sense-and-transmit approach. Therefore, simultaneously achieving energy efficiency and security is difficult. Moreover, imparting smartness to IoT sensors requires additional computations for feature extraction and classification, thus increasing overall energy consumption further. Therefore, it is desired to have sensor side: (1) smartness to alleviate system bandwidth concerns; (2) security to avoid confidentiality/integrity attacks on sensitive sensor data, and (3) energy efficiency in order to enable long-term use without the need for frequent battery change or recharging.

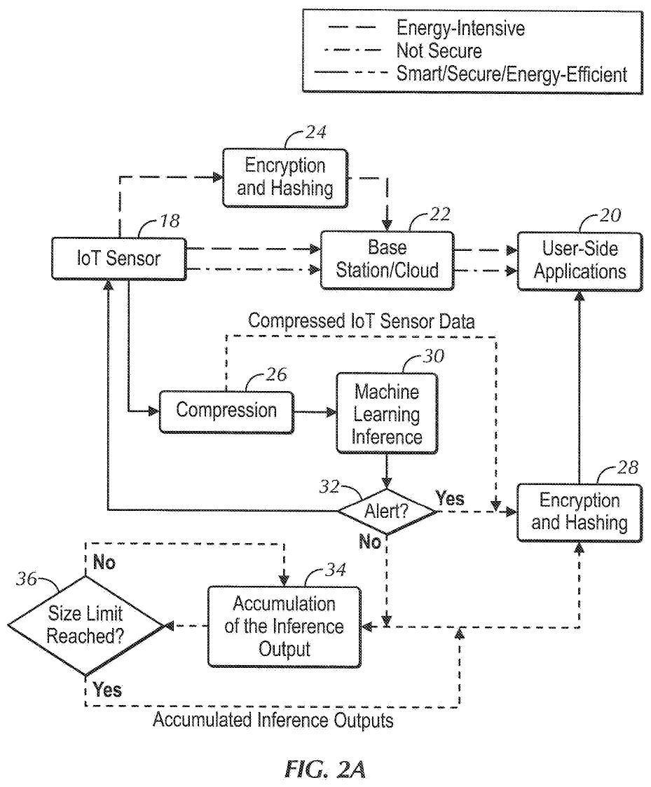

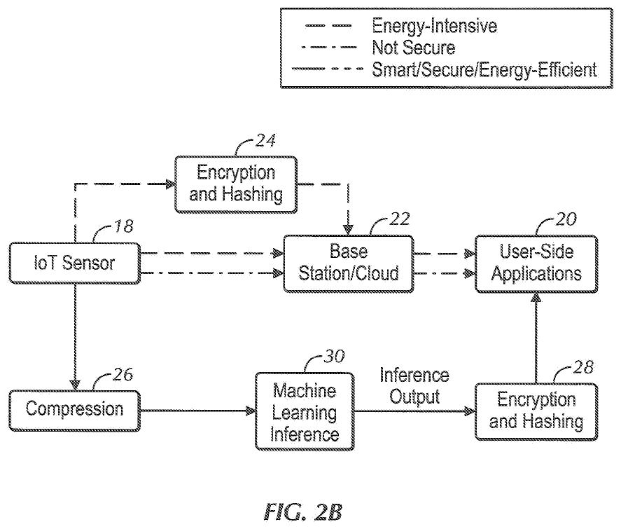

FIG. 2A shows an approach for transmitting and/or processing IoT sensor data from an IoT sensor 18 to user-side applications 20 based on alert notification and FIG. 2B shows an approach for transmitting and/or processing IoT sensor data from an IoT sensor 18 to user-side applications 20 based on continuous notification, though it is noted these approaches are only illustrative and not limiting. Traditionally, one or more base stations and cloud servers 22 are used for processing and/or decision-making.

Alert notification is applicable to scenarios when the base station 22 needs to be notified when a rare event, such as arrhythmia, has been detected through on-sensor inference. Continuous notification is applicable to scenarios when the base station 22 needs to be continuously notified of the on-sensor inference outcome, e.g., in the case of human activity detection. In both approaches, sending all the raw data from a traditional sense-and-transmit IoT sensor 18 to the base station or cloud 22 is energy-intensive (even more so when the data are encrypted/hashed 24 before transmission). When no encryption/hashing 24 is employed, the data is vulnerable to eavesdropping and confidentiality/integrity attacks. The propagation of decisions from the base station or cloud 22 to user-side applications 20 can also be energy-intensive and vulnerable to manipulation.

The solid and short-dashed arrows depict the paths taken by an approach according to an embodiment of the present invention. The dashed lines indicate an “alert” scenario and the solid lines depict a “continuous” scenario.

For alert notifications (FIG. 2A) according to an embodiment of the disclosed approach, IoT sensor data is compressed 26. The compressed sensor data may directly be encrypted/hashed 28 before transmitted to user-side applications 20. Preferably, the compressed data is directed to a machine learning inference 30, which queries whether there is an alert 32. If yes, the data is then encrypted/hashed 28 and transmitted to the user-side applications 20. If no, the inference output is accumulated 34 until a size limit is reached 36. Then, the accumulated inference outputs are encrypted/hashed 28 and transmitted to the user-side applications 20.

For continuous notifications (FIG. 2B) according to an embodiment of the disclosed approach, IoT sensor data is compressed 26 and then a machine learning inference 30 is performed. The inference output is then encrypted/hashed 28 and transmitted to the user-side applications 20.

It is to be noted the disclosed approach allows for transmitting IoT sensor data directly from the IoT sensor 18 to the user-side applications 20, though cloud servers and/or base stations 22 may still be utilized depending on the embodiment.

Within the sensor node 18, the data can be compressed/processed using two different approaches: direct computations on compressively-sensed data or CSP. In the former, signal processing is directly performed on a compressed representation of data, whereas in the latter, signal processing and compression are carried out in the Nyquist domain. Inference is performed in the compressed domain itself, based on the IoT application of interest. The inference outcome determines what data is transmitted further and how often. This enables simultaneously achievement of (1) smartness through machine learning inferences, (2) security through encryption and hashing, and (3) energy efficiency through both compression and machine learning inferences, which enable a drastic reduction in the amount of data transmitted to the base station.

IoT Sensors 18 and Nonlimiting Example Applications

IoT sensors 18 measure a physical quantity and communicate with other sensors, actuators, and applications by utilizing the Internet. Owing to their Internet connection, possibly through gateway devices, IoT sensors 18 no longer cater to a single functionality, but are integrated into systems with artificial intelligence (AI) capabilities. With this technological transformation, IoT systems become capable of processing the data and making a decision, thus imparting smartness to the system.

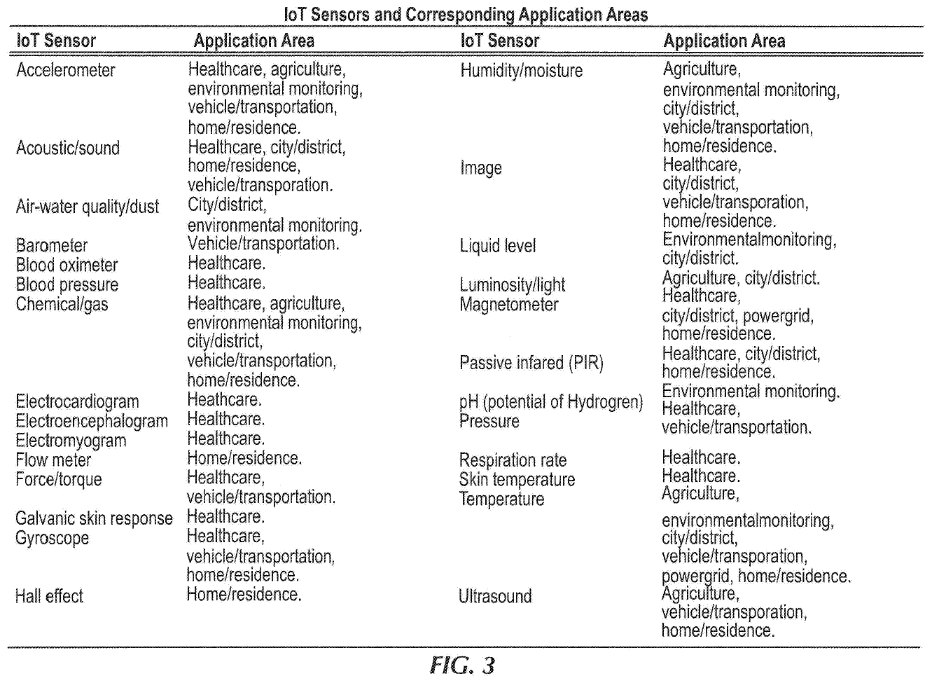

These capabilities have given rise to myriad applications 20. For example, healthcare applications include monitoring the health indicators of the user to avoid accidents, detect diseases at an early stage, enhance patient care, infuse precise amounts of medication into the body, and support patient treatment. Agricultural applications include the monitoring of animals, assessing their breeding, and analyzing agricultural production. Environmental monitoring applications track the chemical properties of air, measure humidity/temperature/water levels, and anticipate/analyze natural/human-made hazards. City/district applications facilitate utilization of parking lots, regulate traffic, assess weather conditions, trace environmental pollution, and ensure safety of the city. Vehicle/transportation applications automate payment for parking, toll, etc., anticipate/report traffic accidents, provide information on road topology, and enable the driver to navigate to a specific location. Power grid applications monitor the use of electricity, automate energy processes, analyze reliability, and enhance security/privacy of the overall system. Home/residence applications track physiological signals from the human body and obtain data from embedded sensors in the environment to guide the user towards a healthier, safer, and more comfortable lifestyle. Some nonlimiting examples of IoT sensors 18 and their corresponding application areas 20 are listed in the table in FIG. 3. It gives an inkling of the wide applicability of these sensors.

Data Compression 26

Compression 26 decreases data size while aiming to preserve the information embedded in the data. It reduces system resources devoted to processing, inference, storage, and transmission. This leads to energy and storage benefits. This is especially beneficial to systems with severe resource constraints.

Compressive sensing is one such method. In compressive sensing, the data is randomly projected to the compressed domain and, when needed, the compressed data is reconstructed by exploiting sparsity in a secondary basis (i.e., the basis in which the data is sparse). The original data is retrieved if the random projection matrix and dictionary of the sparse basis are incoherent with each other. In this technique, although the compression stage has low computational cost (just one matrix multiplication), data reconstruction is very energy-intensive (three to four orders of magnitude more than data compression) and thus not feasible on the sensor. To find a way around this problem, signal processing and inference may be directly performed on compressively-sensed data, thus eliminating the need for data reconstruction. Since machine learning systems often rely on distance metrics for classification, they focus on minimizing the inner product error in the case of the uncompressed and compressed feature vectors.

CSP is another data compression approach. It processes the data in the Nyquist domain, but also relies on random projections to reduce system resources. This reduces the inner product error even more, thus improving classification accuracy of inference.

Feature Extraction and Inference 30

The feature extraction stage extracts informative values (i.e., features) from the data collected through IoT sensors 18. The feature extraction process may lead to linear or nonlinear features. The inference stage 30 takes these extracted features as input and makes a decision by utilizing previously-trained machine learning models. These models can be derived using any machine learning system. The choice of system depends on the resultant model complexity, available energy/storage resources, and data characteristics.

Random forest is a preferable machine learning system in the context of embedded devices. It is an ensemble of decision trees, each of which votes for the class of the given data instance. The trees are generated using a randomly-selected subset of features and sampling of training data instances with replacement. This typically leads to high classification accuracies and robustness to outliers/noise, simplicity, and fast inference. Hence, it is a favored option for resource-constrained applications.

Adaptively boosted (AdaBoosted) decision tree is another ensemble method that can be used by embedded systems for classification purposes. It makes predictions based on a weighted sum of votes from decision trees that are used as weak classifiers. The classifier derived from this system can typically achieve high classification performance with low computation energy.

K-means is another machine learning system for clustering analysis. It iteratively updates the cluster centers during training. To make a prediction, it compares an incoming instance against the existing cluster centers based on similarity metrics, such as cosine similarity, and then predicts the label based on the most similar cluster center.

Encryption and Hashing 28

IoT sensors 18 collect sensitive information, thus requiring meticulous conservation of the security principles: confidentiality, integrity, and availability. These principles can be secured through encryption and hashing 28. Advanced Encryption Standard (AES) and Secure Hash Algorithm (SHA) are preferable encryption and hashing methods.

AES uses a symmetric key for encryption and decryption. It encrypts 128-bit blocks with 128-, 192-, or 256-bit keys. It includes four main operations: SubBytes, ShiftRows, MixColumns, and AddRoundKey. These operations are repeated in multiple rounds based on the number of bits in the key. The last round does not involve the MixColumns step and outputs the ciphertext (i.e., encrypted plaintext). The size of the ciphertext is equal to the plaintext, which is a multiple of 128 bits.

SHA-3 is the latest hashing technique used for integrity checking. It uses the KECCAK algorithm and produces fixed-length outputs (160, 224, 256, 384, or 512 bits). SHA-3 prevents malicious manipulation of data. If the data is tampered with, the hash algorithm gives a different output and reveals the manipulation.

Data Transmission

Communication and data transmission between IoT sensors 18 and the base station 22 are crucial to the operation of the IoT system. Many data transmission protocols are available. The choice depends on energy/storage resources, latency tolerance, and data size/frequency.

Bluetooth Low Energy (BLE) is one of the most widely used data transmission protocols. It provides short-range communication in the 2.4 GHz Industrial Scientific Medical (ISM) band. It uses master and slave devices. Each master has multiple slaves. The master is responsible for determining the listening schedule for and providing connection/frequency information to the slave. Except for waking up at specific time intervals to listen to the packet, slaves stay in the sleep mode. This saves system energy.

The medical implant communication service (MICS) band is a widely used communication band that supports communication between low-power implanted medical devices and external monitoring or control equipment. The 402 to 405 MHz MICS band offers reasonable propagation characteristics for signals within and around human bodies. The introduction of MICS has led to the advent of new medical applications, where various wireless nodes in, on, or around a human body can collaborate to monitor vital signs.

System Overview

As described earlier, smartness, security, and energy efficiency are vital objectives of IoT sensor design. Previously, these objectives were typically targeted individually due to the trade-offs involved in navigating between security and energy efficiency or smartness and energy efficiency. All three objectives have not been targeted simultaneously previously.

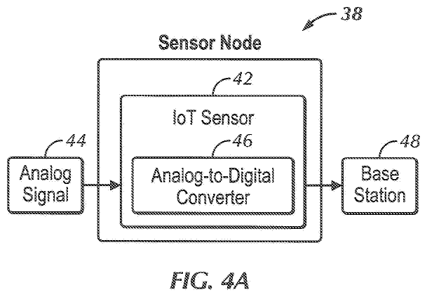

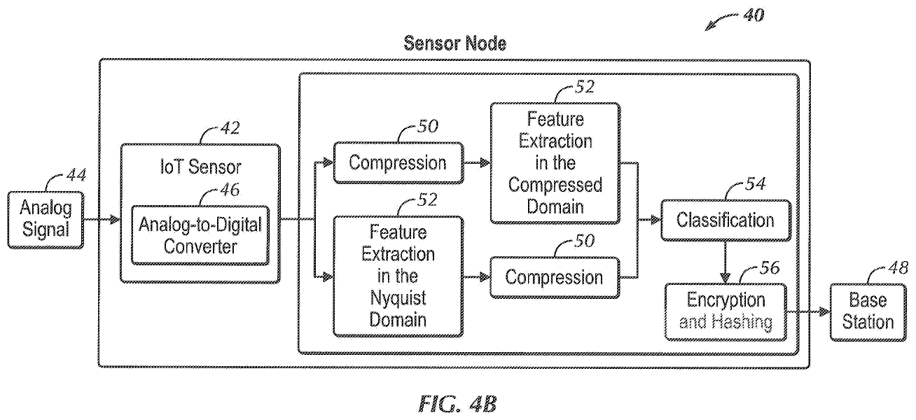

Introduced herein is a novel IoT sensor architecture to address the above challenges. FIG. 4A shows a conventional sense-and-transmit IoT sensor architecture 38 and FIG. 4B shows the disclosed smart, secure, and energy-efficient IoT sensor architecture 40.

The conventional sensor architecture 38 includes an IoT sensor 42 that converts an analog signal 44 into a digital signal via an analog to digital converter 46, which is then transmitted to a base station 48.

In contrast to the traditional IoT sensor 42, embodiments of the disclosed architecture 40 include various additional processing stages (i.e., compression 50, feature extraction 52, inference 54, and encryption/hashing 56, to be discussed in further detail below). These stages are performed via one or more processors and stored in memory, the processors and memory included on the sensor node 40. The one or more processors could be a central processing unit (CPU) or a field programmable gate array (FPGA), as nonlimiting examples.

These additional processing stages provide two data compression/feature extraction 50/52 options to the users. One of these options compressively senses the data and carries out the remaining operations in the compressed domain, whereas the other option employs signal processing in the Nyquist domain and then carries out the compression.

Following data compression 50 and feature extraction 52, the architecture performs classification 54 for continuous and/or alert notification scenarios. In the alert notification scenario, the system carries out classification 54 to assess whether an alert needs to be issued (e.g., arrhythmia detected by a smart electrocardiogram sensor or seizure detected by a smart electroencephalogram sensor). In case of an alert, the data (encrypted and hashed 56) is transmitted to the base station 48. In the no-alert condition and continuous notification scenario, the classification results are accumulated, stored in the memory, and sent to the base station 48 (after being encrypted and hashed 56) at specific time intervals.

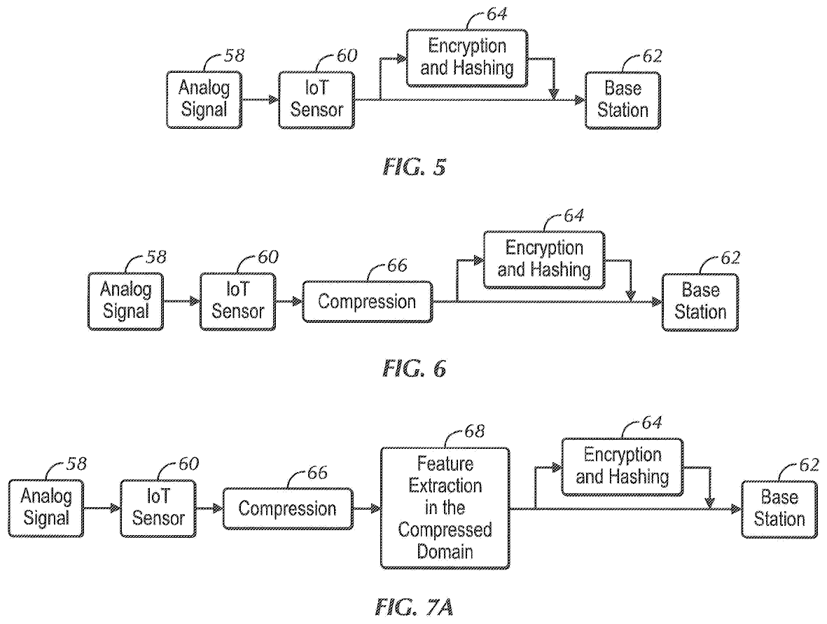

The architecture 40 allows flexible deployment of the processing stages. Depending on the application and its objectives, a relevant subset of the processing stages can be chosen. FIGS. 5 and 6 show paths through the architecture that correspond to sense-and-transmit and sense-compress-transmit approaches, respectively, with an option to employ cryptographic techniques. For sense-and-transmit, an analog signal 58 is transmitted to an IoT sensor 60, which is converted to a digital signal that is transmitted to a base station 62. Encryption and hashing 64 may be employed. For sense-compress-transmit, the digital signal is compressed 66 before transmission to the base station 62.

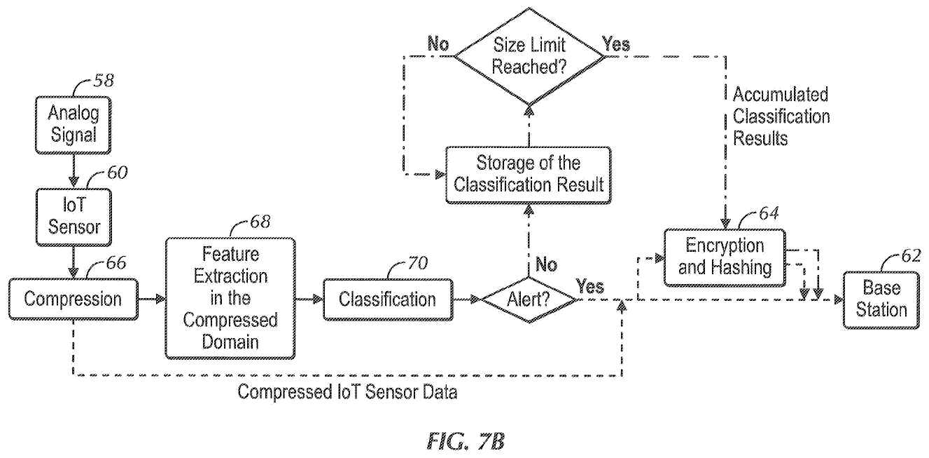

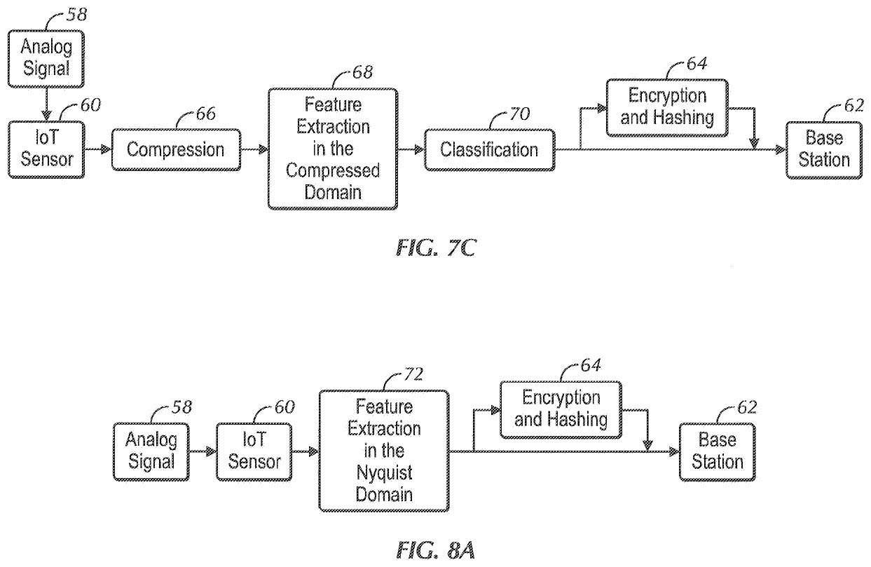

FIGS. 7A-C show different paths corresponding to compression and data processing in the compressed-domain with options to carry out classification and encryption/hashing. The approach in FIG. 7A extracts features 68, but does not carry out classification 70. This approach transmits the features extracted from the input data. Since it does not do classification, it cannot raise an alert. Due to the computational load for signal processing and transmission of the feature vector corresponding to each input vector, the approach in FIG. 7A does not offer much energy efficiency. Thus, the energy consumption of the paths shown in FIGS. 7B and 7C are analyzed instead for alert and continuous notification scenarios, respectively. By doing more (i.e., classification 70), they counter-intuitively require less energy since the output of the classification stage only has a few bits per input vector. This dramatically cuts down on the amount of data transmission to the base station 62. To be discussed further below, since transmission energy dominates sensor energy, this provides a huge energy benefit.

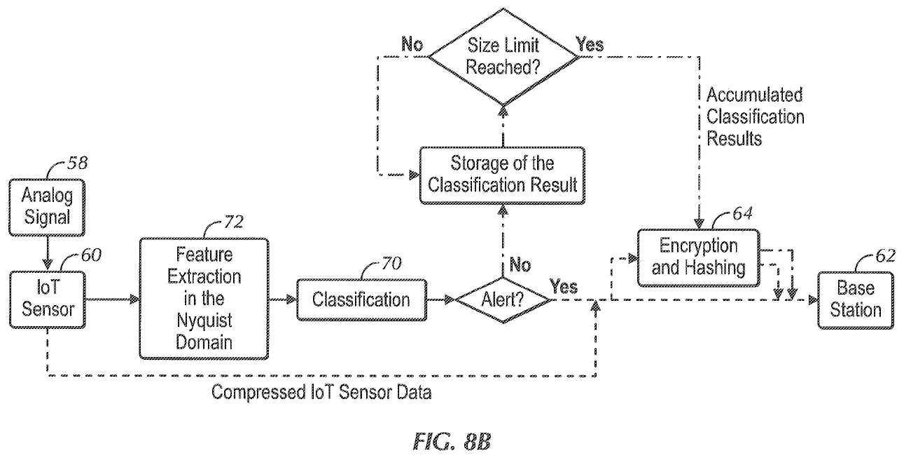

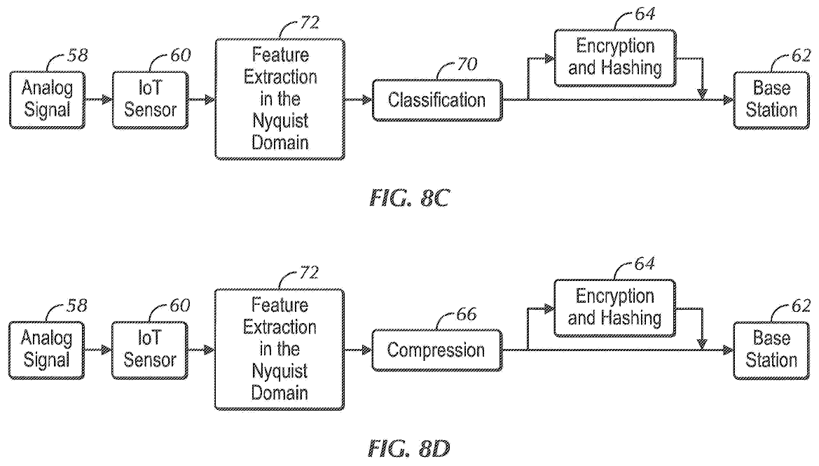

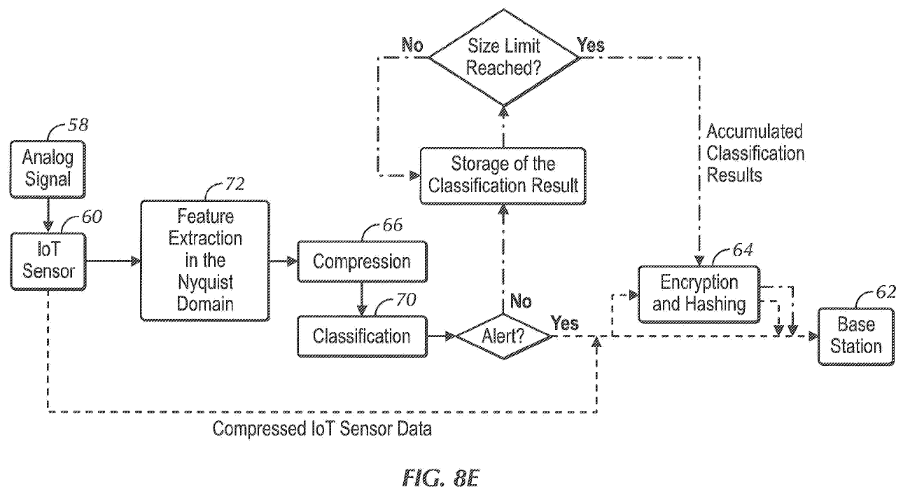

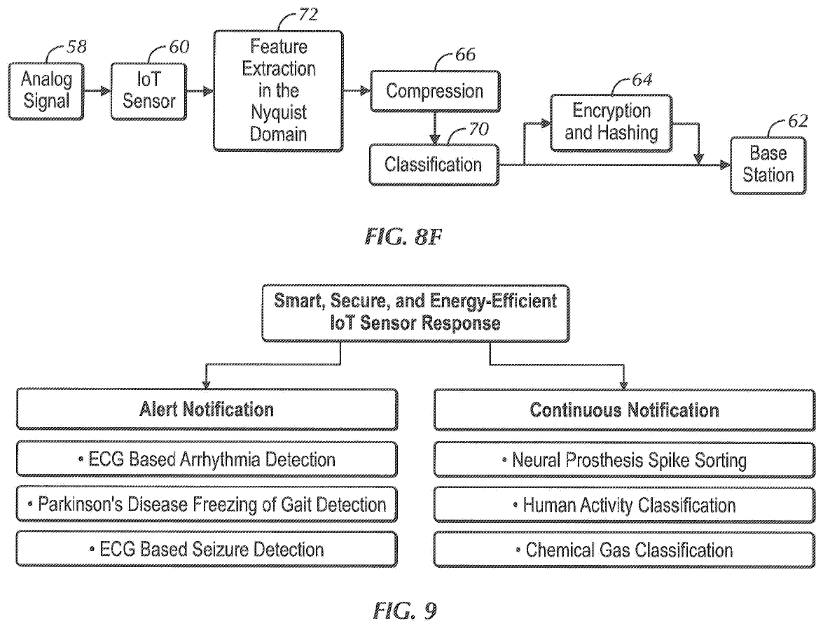

FIGS. 8A-F shows various architectural paths that can be used in a reduced architecture based on compressed signal processing. Here, feature extraction 72 is performed in the Nyquist Domain. As in the case of FIG. 7A, FIGS. 8A and 8D perform signal processing without utilizing classification. This results in increased energy consumption and since these approaches are only useful when energy is not a concern, they are not a focus here. The approaches shown in FIGS. 8B and 8C, respectively alert and continuous notification scenarios, depict uncompressed embodiments of the approaches shown in FIGS. 8E and 8F, respectively. To be discussed further below, the energy consumption of both the uncompressed and compressed versions are analyzed.

Energy Modeling

Embodiment of the disclosed IoT sensor architecture 40 include additional processing components useful for energy-constrained sensor nodes. Since performing more operations on the sensor node, yet claiming energy efficiency, is counter-intuitive, it must be demonstrated that this is a viable claim through detailed energy modeling. Thus, energy bonus/overhead analyses of each additional processing component (shown in FIG. 4B) is important for assessing the applicability of the proposed architecture. With the inclusion of new processing components in FIG. 4B: (1) the total number of multiply-accumulate (MAC) operations and static random access memory (SRAM) accesses are impacted; (2) the amount of data that requires encryption/hashing and transmission to the base station is altered, and (3) classification incurs extra energy. In order to make a fair comparison between the traditional sense-and-transmit approach and various embodiments of the disclosed architecture, the energy of all these versions is modeled as shown in Eq. (1):

E_{Total}=E_{MAC}+E_{SRAM}+E_{Cl}+E_{Enc}+E_{Hash}+E_{Tr} (1)

E_{MAC} and E_{SRAM} respectively refer to MAC operation and SRAM access energy consumed in the compression block 50 and feature extraction block 52. E_{MAC} and E_{SRAM} do not include MAC/SRAM energy consumption in the remaining blocks (i.e., classification 54, encryption/hashing 56, and transmission to the base station 48). The MAC/SRAM energy in these blocks are accounted for in their respective energy models: E_{Cl}, E_{Enc}, E_{Hash}, and E_{Tr}, which represent classification, encryption, hashing, and transmission energy, respectively. The energy for analog-to-digital conversion is ignored since it is insignificant relative to the other energy components.

The analysis is based on the 130 nm technology, but valid for any other CMOS technology. Measurements from a 130 nm CMOS IC indicate 11.8 pJ and 34.6 pJ energy consumption, respectively, for a 32-bit MAC operation and access of data from a 32 kB SRAM. E_{MAC} is computed by multiplying the unit MAC operation energy with the total number of MAC operations in the compression block 50 and feature extraction block 52. For example, the multiplication of M×N and N×K matrices with 32-bit entries requires (M·N·K) MAC operations, hence, a total of (11.8·M·N·K) pJ of energy. Likewise, E_{SRAM} is obtained by multiplying the energy of a single SRAM access with the total number of SRAM accesses. In the matrix multiplication example, the SRAM is accessed (2·M·N·K) times. This leads to an energy consumption of (34.6·2·M·N·K) pJ.

To obtain E_{Cl}, the classification energy for the random forest, AdaBoosted decision tree, and the K-means processes are modeled. The resulting classifier of the random forest process includes a large number of decision trees, each of which employs thresholds on feature values for branching. This computation starts with a unit thresholding energy of 4.09 fJ obtained based on an 8-bit binary tree comparator in 180 nm CMOS technology. Since this does not match the technology or bit-width assumed for the other blocks, four 8-bit comparators are used to design a 32-bit comparator and Dennard scaling is used to scale results to the 130 nm CMOS technology. Dennard scaling from 180 nm to 130 nm decreases capacitance and voltage by a factor of 180/130. Thus, it entails dividing the overall classification energy by (180/130)³. Each tree node in a random forest consumes energy for unit thresholding and two associated SRAM accesses (one for the threshold value and the other for the pointer to the next node) for a single comparison. The overall E_{Cl} for the random forest model is obtained by multiplying this energy per tree node comparison, the maximum tree depth, and the total number of trees. This is a conservative estimate since not every tree is traversed to its full depth for a given data instance.

The classifier derived from the AdaBoosted decision tree process relies on comparisons (at tree nodes and final output) and multiplications (between tree outputs and their weights) to make a prediction. The energy is modeled for these two parts separately, and the results are accumulated to obtain the final E_{Cl} for the AdaBoosted decision tree model.

The classifier derived from the K-means process involves the inner product calculations between each incoming instance and the cluster center vectors for similarity analysis. Thus, K−1 comparisons are needed to obtain the best prediction among the K cluster centers. The incurred energy is accumulated in all these steps to obtain the final E_{Cl} for the K-means model.

To obtain E_{Enc} and E_{Hash}, a gate-level implementation of the encryption (AES-128) and hashing (SHA-3) algorithms is used in 65 nm CMOS technology. The energy is scaled by a factor of (130/65)³ to make it compatible with the 130 nm CMOS technology.

To obtain the transmission energy, E_{Tr}, the focus is on the amount of data that needs to be transmitted to the base station 48. In case of an alert, the compressed data is transmitted immediately, whereas under the no-alert condition, the classification results are accumulated and then transmitted. With a BLE connection that sends up to six packets, each containing 20 B of data, the required number of packets and connection intervals needed for transmission are calculated. Then, using the current and timing measurements of various BLE stages, i.e., wake-up, preprocessing, pre-listening, listening, pre-transmission, transmission, post-processing, pre-sleep, and sleep, E_{Tr} is obtained for BLE based on but not limited to TI CC2650 module measurements. Since BLE has built-in encryption/hashing, encryption/hashing energy is not separately taken into account when using BLE. To obtain E_{Tr} for MICS, the 0.51 nJ/bit transmission energy is used based on a transceiver design that accommodates the signal loss due to the shadowing effects from human bodies. It achieves very high modulation accuracy, sensitivity, and interference robustness. However, in the MICS case, encryption/hashing energy needs to be separately added.

Experiment Results

Presented below are experimental results for embodiments of the smart, secure, energy-efficient IoT sensor architecture when applied to various datasets.

Datasets

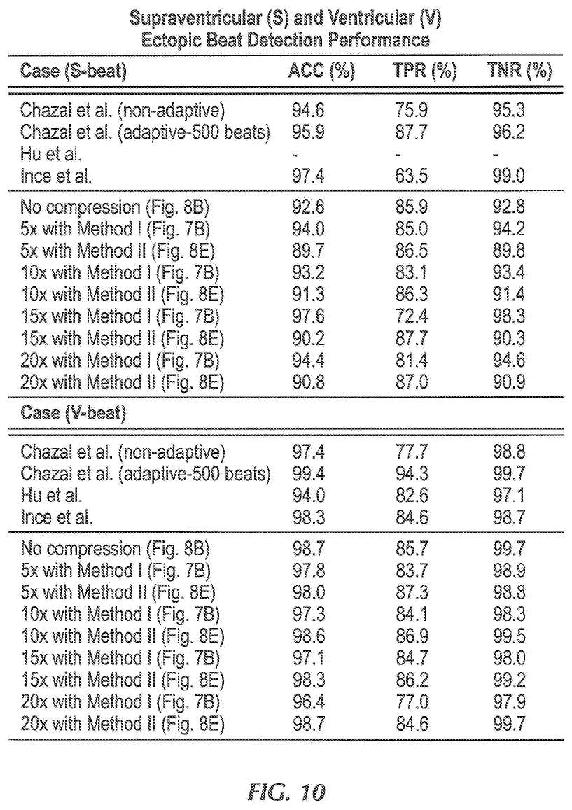

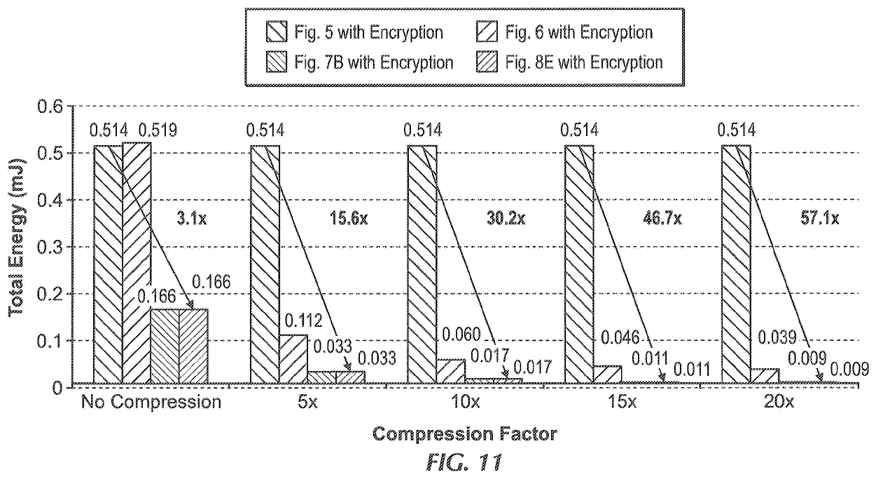

The disclosed sensor architecture is versatile and applicable across IoT applications. Its effectiveness is evaluated based on six IoT datasets. As shown in FIG. 9, these datasets are divided into two groups based on their requirements: alert notification or continuous notification. Arrhythmia, freezing of gait, and seizure detection applications require immediate action to minimize/eliminate unwanted consequences (e.g., injury, brain damage, heart attack, death). These are called alert conditions. The alert notification system informs the base station/server when the event of interest occurs. For non-alert conditions, the proposed system sends information to the base station/server at specific time intervals to certify that the system is up and running, without the need to trigger alert notification.

The MIT-BIH arrhythmia database includes ECG measurements that are utilized for arrhythmia detection. The UCI Daphnet Freezing of Gait Dataset is based on acceleration sensor readings that are used to assess motor blocks in patients with Parkinson's disease. The neural prosthesis dataset is used for spike sorting. The UCI Daily and Sport Activities Dataset includes accelerometer, gyroscope, and magnetometer measurements that are used for human activity detection. The CHB-MIT Scalp EEG Database includes EEG measurements that are used for epileptic seizure detection. The UCI Gas Sensor Array Drift Dataset includes metal-oxide gas sensor measurements for chemical gas classification.

The experimental results are presented for each of the six datasets next. The first five datasets contain linear features. These features can be extracted via matrix-vector multiplications, where both the compressed-domain feature extraction technique and the CSP technique can be directly applied to cut down on computation energy. 57.1-379.8× energy reduction was achieved for these datasets. Then, the results for the chemical gas classification dataset that involves nonlinear features are presented. It is shown that even though the nonlinear features cannot be extracted via matrix-vector multiplication, the nonlinearity can be easily handled by existing circuitry in sensor nodes. 912.6× energy reduction was achieved for this dataset.

Alert Notification

The alert conditions in ECG-based arrhythmia detection, freezing of gait detection for Parkinson's disease, and EEG-based epileptic seizure detection applications are an irregular heart rhythm (both supraventricular and ventricular), freezing event, and epileptic seizure, respectively. For arrhythmia, the irregular supraventricular and ventricular heartbeats are detected through the use of classifiers. In order to be in line with previous approaches, the heartbeat annotation of The Association for the Advancement of Medical Instrumentation (AAMI) is used. For freezing of gait, the freezing event is detected through the energy of the accelerometer signal in specific frequency bands with the help of classifiers. Similarly, for EEG-based epileptic seizure detection, the seizure episode is detected by computing the EEG energy in eight different frequency bands and employing classifiers. Overall, the alert notification systems detect irregular events.

ECG based Arrhythmia Detection:

ECG is a physiological signal that provides information on the electrical activity of the heart. It is used to detect irregularities in the cardiovascular system, such as arrhythmia. Arrhythmia is an irregularity of the heart rhythm. If the symptoms are not detected at an early stage, it can lead to cardiac arrest, heart attack, or even death. Continuous ECG monitoring is a method to avoid these severe consequences and enhance patient wellness. Embodiments of the disclosed IoT sensor architecture are directly applicable to the continuous ECG monitoring application. It significantly improves battery lifetime based on on-sensor data compression and classification, and improves security based on encrypted/hashed data transmission.

The MIT-BIH Arrhythmia Database is used to analyze the disclosed architecture in terms of accuracy and energy consumption. This database includes 48 ECG data sections collected from 47 participants. Each data section includes 30 minutes of ECG measurements based on a 360 Hz sampling rate. The ECG measurements include various heartbeat waveforms. AAMI advocates grouping these independently annotated beats into five different classes: normal (N), supraventricular ectopic (S), ventricular ectopic (V), fusion (F), and unknown beat (Q). The AAMI standard is followed in this evaluation. Moreover, due to reduced signal quality issues, four data sections which contain paced heartbeat signals are discarded from the analyses. Since the goal of the disclosed architecture is to detect arrhythmia from ECG heartbeat signals, two different binary classifiers are designed to identify the S and V beats.

The sensor node is designed using the first lead of the ECG signal. It is expected the arrhythmia classification accuracy will improve further when both leads are used. However, the aim here is to show the applicability of the disclosed sensor architecture to various ECG sensors, including single-lead and multiple-lead ones. Therefore, the ECG data from the first lead in the MIT-BIH Arrhythmia Database is used. Also, it is assumed that the ECG sensor provides R-peak positions. This is a reasonable assumption since several on-market sensors are capable of detecting R-peaks. With advancing technology, more sensors are expected to provide this information. However, if this information is not available, then Discrete Wavelet Transform (DWT) is one of the methods for detecting R-peaks. Since DWT can be represented in matrix form, the disclosed architecture is capable of performing R-peak detection.

Following the acquisition of R-peak positions, the ECG signal is divided into 256 sample epochs that cover approximately 0.3 s interval before and 0.4 s after the R-peak. Since these are time-series data, the first 80% of the epochs are used as the training set and the remaining as the test set. From each of these epochs, bandpass filter (BPF) and DWT features are extracted. To obtain the BPF features, the corresponding ECG signal is passed through 30 BPFs (0.5-2.0 Hz, 2.0-3.5 Hz, 3.5-5.0 Hz, . . . , 44.0-45.5 Hz). This involves the convolution operation, which is executed by obtaining the row-shifted version of BPF coefficients. At the end of this row-shift, an M×M convolution matrix is obtained from the array of BPF coefficients (1×M). Then, the convolution matrix is multiplied with the ECG data of the corresponding epoch. At the final step, the inner product of the resulting array is computed and stored in the memory for use in the classification stage. This process is repeated for each BPF. To obtain DWT features, a 6-level DWT is used to capture arrhythmia information. DWT is implemented with a filter bank (consecutive high-pass and low-pass filters). To pass ECG data through high-pass and low-pass filters, the above-mentioned procedure is employed. After each filtering stage, the output is down-sampled by 2×. Since the filters have half the bandwidth of the original signal, aliasing due to down-sampling is avoided. Coefficients obtained through DWT are stored in the memory for use in the classification stage.

Due to its simplicity and fast inference, random forest is used with 100 decision trees having unlimited maximum tree depth for binary classification of S and V beats in the ECG signal (the Weka 3.8.0 platform is used for deriving the machine learning models). The table in FIG. 10 shows the classification accuracy (ACC), true positive rate (TPR), and true negative rate (TNR) for results from the previous work in the upper section, and results based on the disclosed embodiments in the lower section. The results cover the following cases: no compression, 5×, 10×, 15×, and 20× compression based on direct computations on compressively-sensed data (Method I) and CSP (Method II).

A similar performance to those by previous work Chazal et al. is achieved. Their adaptive approach uses a combination of global and local classifiers. It uses the first 500 beats of the training data to train the local classifier. The non-adaptive approach only employs a global classifier. Since the adaptive approach learns more information on the heartbeat types, it results in a higher classification performance. Moreover, compared to results by previous works Hu et al. and Ince et al., a higher TPR is achieved. Since TPR indicates the percentage of correctly classified S and V beats, even with significantly compressed data, the disclosed architecture detects arrhythmia more accurately. Based on the results from the implementations, a similar performance is observed even when the data is compressed.

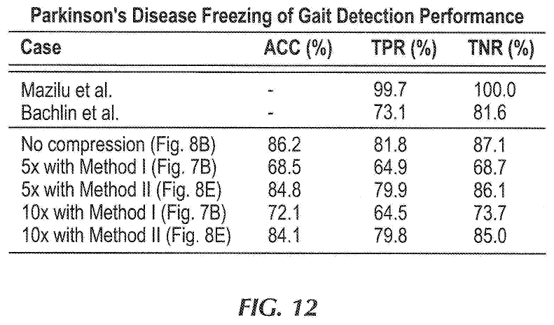

Classification performance is important; however, evaluation of the energy consumption is required to assess the feasibility of the disclosed IoT sensor architecture. The total energy consumption is computed for the different architectural paths, which include encryption and hashing, shown in FIGS. 5, 6, 7B, and 8E. The result for the architectural path depicted in FIG. 8B is shown under the ‘No compression’ case of FIG. 8E, as they are equivalent. The total energy consumption of these paths is compared in FIG. 11 for S-beat and V-beat detection. Overall, a continuing decrease in energy consumption (an increase in energy bonus) is observed with increasing compression factors. This result is as expected. As the data/features are compressed more, the amount of signal processing and transmission packet size both decreases. Machine learning inference decreases the energy consumption by reducing the raw data to a few bits of inference. Since the disclosed system sends the compressed data only when an alert is raised (i.e., when arrhythmia has been detected) and accumulates the inference results for the no-alert case for a more regular data transmission, significant data transmission energy is saved in the no-alert situation. It is observed that up to 57.1× (for the S-beat and V-beat) lower energy is needed to obtain performance comparable to a conventional sense-and-transmit approach.

Freezing of Gait Detection for Parkinson's Disease:

Parkinson's disease affects motor abilities of the patient negatively. One of its consequences is freezing of gait (FoG) due to which the patient loses the ability to move his/her leg temporarily. Sometimes, arms and eyelids are also frozen temporarily. FoG causes injuries to patients, since it arises abruptly and leads to falls. In order to reduce FoG periods and prevent possible falls, previous proposals included a wearable system that provides rhythmic sounds in case a FoG period is detected. This FoG detection application is also directly amenable to implementation with the disclosed sensor architecture, since it employs wearable sensors and requires online FoG detection. Use of wearable sensors necessitates long battery lifetimes, since frequent battery charging or replacement negatively impacts practicality and system adoption.

The UCI Freezing of Gait Dataset is used to evaluate the disclosed architecture in terms of accuracy and energy consumption. The dataset includes accelerometer measurements from ten patients: eight with and two without FoG periods. Personalized FoG detection is implemented. Since two patients do not experience FoG, data from the remaining eight patients is used. The dataset contains measurements from three-axis accelerometers obtained using a 64 Hz sampling rate. The accelerometers are positioned at the shank, thigh, and belt of the patients.

In order to classify the FoG periods, rather than applying thresholding as previously proposed, the random forest is used due to its simplicity and fast inference. To be able to compare classification performance to previously studies, 4 s windows (256 samples) with 0.5 s (32 samples) shifts in between are used. To provide personalized care, the classifier models are built for each patient separately. Keeping in mind having time-series data, the first 70% of the windows are used as the training set and the remaining windows, which do not have any overlap with the training set, are used as the test set. Two BPFs with 0.5-3.0 Hz and 3.0-8.0 Hz bands are used. Since passing a signal through a BPF requires a convolution operation, convolution matrices are obtained for each filter by shifting the rows of BPF coefficients one by one for each row. The corresponding vector of the accelerometer data is multiplied with the two BPF convolution matrices. Then, the inner product of the output array is computed and stored in the memory for use in the classification stage. The above procedure is repeated for each window in the training and test sets. Moreover, the imbalance between classes of the target feature is handled with the SMOTE method. Random forest is used with 100 decision trees having unlimited maximum tree depth for the classification of FoG periods. For each of the eight patients, the classifier model is obtained based on the training set and the performance of the corresponding model on the test set is analyzed. In the classification stage, the features obtained from y-axes of the three accelerometer sensors are used.

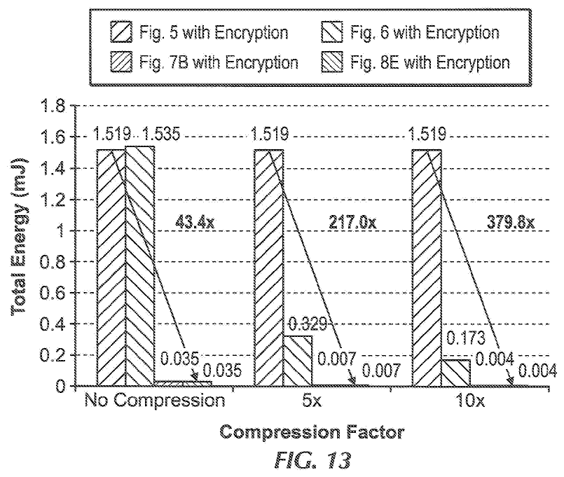

The table in FIG. 12 shows ACC, TPR, and TNR for the results from relevant work in the upper section, and results based on the disclosed embodiments of the present invention in the lower section, which cover the following cases: no compression, 5×, and 10× compression using Method I and Method II. We achieve lower performance compared to a previous study Mazilu et al. However, Mazilu et al. chose training and test sets randomly for a 10-fold cross-validation. Random selection discards the time-series nature of the data and results in correlated data points in the training and test sets. This boosts ACC, TPR, and TNR. In the disclosed embodiments, the time-series nature of data is taken into account by using the first 70% of the data as the training set and the remaining nonoverlapping part as the test set. Thus, a realistic assessment of classifier performance is obtained. Moreover, compared to the more realistic study on online FoG detection Bachlin et al., the disclosed embodiments achieve higher TPR and TNR values. Bachlin et al. used thresholding; however, the disclosed embodiments employ machine learning systems to detect FoG. Machine learning systems are able to identify more complex patterns and thus result in higher classification performance.

Comparable classification performance is observed for no compression and Method II with 5× and 10× compression factors. However, Method I did not achieve as high a performance as Method II. Method I performs feature extraction in the compressed domain. This introduces a small amount of error in the inner product values. In this case, these errors were consequential whereas they were not in the case of arrhythmia detection.

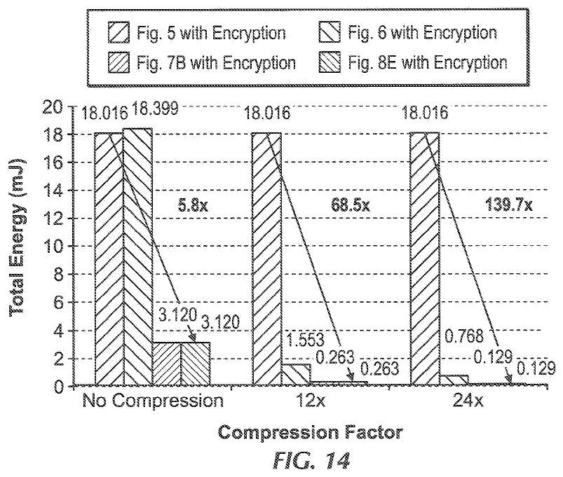

Energy consumption analyses is also performed. FIG. 13 shows the total energy consumption of the architectural paths, which include encryption and hashing, shown in FIGS. 5, 6, 7B, and 8E. Method II, with compression factors of 5× and 10×, reduces energy consumption by 217.0× and 379.8×, respectively, relative to a conventional sense-and-transmit case. Thus, the disclosed architecture results in a huge energy bonus. As pointed out earlier, the cryptographic techniques are already integrated into BLE operations.

EEG based Seizure Detection:

Epilepsy is a neurological disorder that can lead to an abrupt loss of consciousness and body convulsion. It currently affects 4-5% of the world population. Abrupt epileptic seizure onsets pose physical risks to epilepsy patients. A continuous seizure detection system can help mitigate these risks and enhance the quality of healthcare. Such a system also needs to be secure and energy-efficient.

A previous study Lu et al. conducted a detailed performance comparison of Method I and Method II for EEG epileptic seizure detection. They used the data collected from three epileptic patients present in the CHB-MIT Scalp EEG Database, for performance evaluation. They partitioned the EEG signal streams into three-second signal epochs with a two-second overlap. They used eight BPFs (sequentially covering the 0-24 Hz range with 3 Hz individual pass-band widths) to extract the spectrum energy features from the data epochs. They used an AdaBoosted decision tree classifier for seizure detection.

There are three classifier performance metrics that are important for this application: sensitivity (same as TPR), latency, and false alarm rate. Sensitivity specifies how well the inference model can capture seizure onsets. Latency denotes the number of seconds between a physiological seizure onset and system detection. The false alarm rate specifies the number of false predictions divided by the total length of EEG recordings in hours.

Energy consumption analyses is conducted for this application of the disclosed architecture. The EEG signals are captured by an 18-channel EEG sensor front-end. The sampling rate for each channel is 256 Hz. For the energy evaluation of the feature extraction step, two distinct steps are considered that require MAC operations and SRAM accesses. The first step is linear filtering by BPFs that involves matrix-vector multiplication. The second step is energy accumulation based on the inner product of the post-filtered 144-dimensional (8 features per channel×18 channels) signal vector with itself. The energy from these two steps is summed to model the feature extraction energy. The classification energy is modeled based on an AdaBoosted decision tree parameter set that achieves the best performance. Hence, the maximum number of weak classifiers is set to 200. A weak classifier is a shallow decision tree with a maximum depth of three. As a result, the worst-case classification energy corresponds to a data instance that requires 601 comparisons (200 trees×maximum tree depth of 3+1 final comparison) and 200 multiplications (one per tree).

The energy results are summarized in FIG. 14. Embodiments of the disclosed architecture cut the energy consumption by 5.8× for the no-compression case (i.e., when signals are in the Nyquist domain). This ratio increases to 68.5× and 139.7× for 12× and 24× compression factors, respectively.

Continuous Notification

Continuous notification sensors provide regular feedback to the user. Their data transmission to the base station does not depend on the classifier output outcome. The human activity classification, neural prosthesis spike sorting, and chemical gas classification applications fall into this category. For human activity classification, the activities are determined through inputting the collected electromechanical signals to the machine learning system. For neural prosthesis, the spikes are classified by inputting the DWT features of neural spikes to a clustering process. Similarly, the chemical gases are determined by inputting the combination of linear and nonlinear features to the corresponding classifiers.

Human Activity Classification:

State-of-the-art body-wearable sensors enable myriad daily applications, such as daily activity monitoring, sleep status analysis, stress detection and alleviation, and pervasive disease diagnosis. Such a sensor has to be both secure, to protect a user's privacy, and energy-efficient, to increase the battery lifetime for a user's convenience and satisfaction. This section targets human activity classification with body-worn miniature inertial sensor units. This application enables its users to have a direct, fine-grained visualization of their life logs.

The UCI Daily and Sport Activities Dataset is used to evaluate the disclosed architecture for this application. This dataset contains 19 different activities that comprehensively cover a wide range of daily routines: sitting (1), standing (2), lying on back (3) and right side (4), ascending (5) and descending stairs (6), standing still (7) and moving around (8) in an elevator, walking in a parking lot (9), walking on a treadmill with a speed of 4 km/h in flat (10) and 15-degree inclined positions (11), running on a treadmill with a speed of 8 km/h (12), exercising on a stepper (13), exercising on a cross trainer (14), cycling on an exercise bike in horizontal (15) and vertical positions (16), rowing (17), jumping (18), and playing basketball (19). The data is collected from four female and four male participants. Each participant wears five body-worn miniature inertial sensor units. Each tracker contains a tri-axial accelerometer, a tri-axial gyroscope, and a tri-axial magnetometer. The sampling rate per sensor per axial is 25 Hz. Each activity contains five minutes of data per participant, and is divided into 5-second intervals. Therefore, there are 480 data intervals per activity.

The classifiers for this application are trained next. Previous work Altun et al. extracted 1170 features from each 5-second data interval, and then reduced the feature dimension to 30 through principal component analysis. They performed a leave-one-out (L1O) analysis for this application. In this analysis, a new machine learning model is trained in each L1O iteration based on the data from seven participants (7980 training instances=60 vectors per person per activity×7 persons×19 activities) and tested on data from the remaining participant (1140 testing instances =60 vectors per person per activity×1 person×19 activities). Data from each participant is used only once for testing. The average accuracy over the eight iterations denotes the final performance. They achieved the highest accuracy of 87.6% with a support vector machine (SVM) classifier. The system delay is five seconds because of the 5-second data interval required for feature extraction.

In order to decrease the system delay and improve classification performance, a 0.04 s data interval is employed and the first 80% of the participants' data (7296 training instances=48 vectors per person per activity×8 persons×19 activities) is utilized for training and the remaining (1824 testing instances=12 vectors per person per activity×8 persons×19 activities) for testing. 96.0% accuracy is achieved using the random forest algorithm (100 trees with a maximum tree depth of 10). This shows that the disclosed architecture is approximately 8% more accurate and 125× faster than the design in Altun et al.

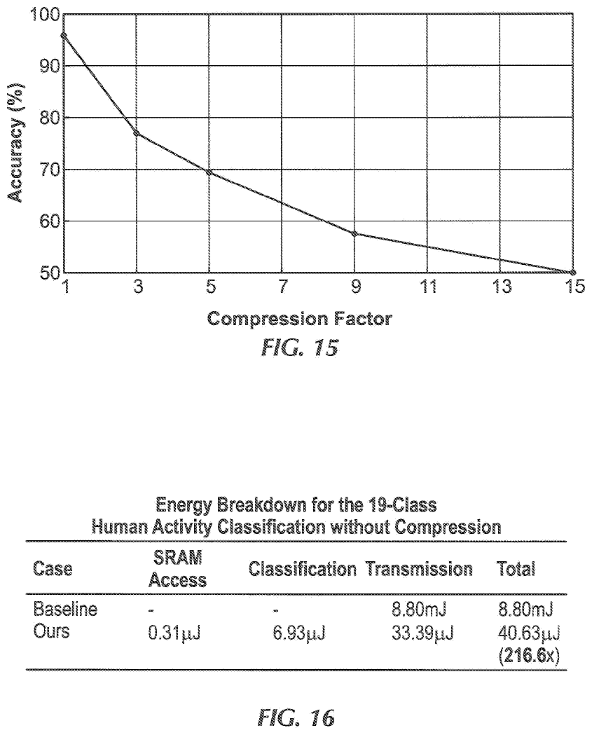

In FIG. 15, the classification accuracy is shown for various compression factors. The classification accuracy drops from 96.0% in the Nyquist domain to 69.7% and 50.1% with 5× and 15× compression factors, respectively. This significant performance degradation may be explained by the fact that the 19-class classification task is very challenging, thus more sensitive to information loss caused by compression.

Due to the significant accuracy degradation in the compressed domain, energy analyses for this application is only performed in the Nyquist domain. Worst-case random forest classification energy for an input data instance is considered that requires 1K comparisons (100 trees×a maximum tree depth of 10). The sensor transmission protocol is assumed to be BLE. Thus, encryption and hashing energy is not modeled separately as BLE already incorporates them.

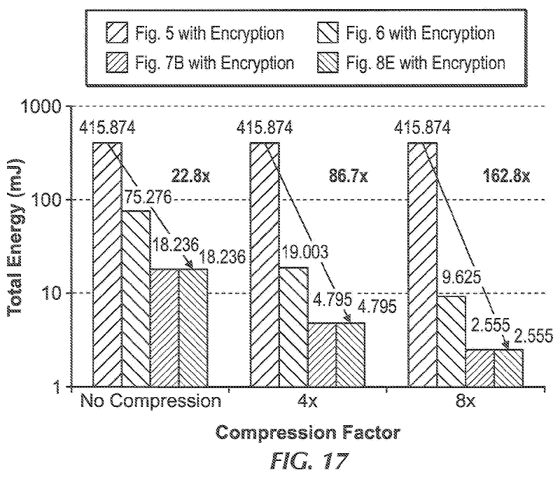

The results are summarized in the table in FIG. 16. Its columns correspond to the method, SRAM access energy for local data instance storage, classification energy, transmission energy, and total energy consumption for this application. The first row shows the energy values for the traditional sense-and-transmit approach. The second row shows the energy values for the disclosed approach. 216.6× energy reduction is achieved for this application.

Neural Prosthesis:

Neural prosthetic systems enable external devices to collect, analyze, and respond to the neural activities in human brains through prosthetic implants. These systems can alleviate treatment-resistant depression and chronic pain, Alzheimer's disease, post-traumatic stress disorder, traumatic brain injury, speech disability, and sustained spinal cord injury. Due to the high risk of surgery, the lower the energy computation (and hence longer the battery lifetime) of the prosthetic implants, the better.

The disclosed sensor architecture is evaluated for neural prostheses that detect and sort neural spikes. To address the energy and bandwidth constraints, the spike analysis process is carried out on biomedical implants. The spike analysis process has five sequential stages: analog-to-digital conversion (ADC), spike detection, spike alignment, feature extraction, and spike classification. The classification results are transmitted to an external controller via low-power transceivers that operate in the MICS band.

For neural prostheses, previous work Lu et al. conducted a detailed performance comparison of Method I and Method II. They used spike records for performance evaluation and simulated the records for three neural spike classes under 18 different neural noise levels. They extracted DWT features and used a K-means classifier for spike sorting. On an average, Method I (Method II) can correctly classify 87.8% (87.8%) of the spikes without compression with a K-means classifier. This accuracy slightly drops to 83.9% (85.6%) and 82.5% (83.2%) with 4× and 8× compression factors, respectively.

Energy consumption analyses is conducted for this application. The ADC front-end samples signals at 24,000 Hz with 32 bits per sample. The system uses an amplitude threshold method (a 4δ rule based on standard deviation of the background noise) for spike detection. The associated comparisons and SRAM accesses for the spike detection energy calculation are considered. Whenever a spike is detected, the system keeps track of 64 samples in one data segment for further analysis. The average frequency of spike occurrence is 51.9 neural spikes per second. These spikes incur subsequent feature extraction, classification, and transmission energy consumption. In the Nyquist domain, 64 DWT coefficient features are extracted from each spike data segment for subsequent classification (the number can be reduced in the compressed domain using Method I and Method II to reduce computation energy). Both the MAC operations and SRAM accesses are considered due to the matrix-vector multiplications for DWT feature extraction energy calculation. Since this application needs a three-class classification, the energy for a K-means classifier that contains three cluster centers is modeled. Data transmission is based on MICS transceivers. For security purpose, AES-128 is used for encryption and SHA-3 is used for hashing, and their energy is also modeled.

The results are summarized in FIG. 17. This figure shows the total energy consumption of various architectural paths, which include encryption and hashing. Without compression (i.e., when Nyquist-domain feature extraction and classification are done in the path shown in FIG. 8F or equivalently FIG. 8C), the disclosed sensor architecture embodiments are able to cut the energy consumption by 22.8× against a conventional sense-and-transmit approach. This ratio further increases to 86.7× and 162.8× with 4× and 8× compression factors, respectively. This shows that the disclosed approaches can provide a huge energy benefit, while at the same time adding security and smartness bonuses.

Chemical Gas Classification with Nonlinear Features:

The impact of the disclosed sensor architecture is analyzed in this section on an application that requires nonlinear features. It is shown that even though such features cannot be extracted via matrix-vector multiplications, the nonlinearity may be easily handled by existing sensor circuitry.

Specifically, the classification of air flow chemical composition using chemical sensors is focused on. Through this application, the feasibility of the disclosed approaches is evaluated for industrial automation/monitoring. Industrial IoT sensors are better than humans at capturing data consistently and accurately. However, transmitting all the raw sensor data to the cloud consumes substantial sensor energy, server storage, and network bandwidth. The disclosed approaches provide a solution to this dilemma.

The UCI Gas Sensor Array Drift Dataset is used to evaluate the disclosed architecture in terms of accuracy and energy consumption. This dataset is targeted at six chemical gases: ammonia, acetaldehyde, acetone, ethylene, ethanol, and toluene. The data is collected by 16 commercially available chemical sensors in a controllable test sensing chamber. Each sensor yields a time series of measurements. Eight features are extracted from each time series. The feature vectors and their labels are placed in ten data batches prior to upload to UCI. Batch 1 is used for training (first two months of data) and batch 2 is used for testing (next five months of data) for two reasons: (1) to avoid the sensor degradation phenomena in later data batches; and (2) both the training and testing datasets are needed to contain labels for all six target gases to enable a more comprehensive analysis. One handicap in dealing with this dataset is that the raw data is not available, just the feature vectors.

This problem is first analyzed in the Nyquist domain. A classification accuracy of 75.6% is achieved using random forest (100 trees with a maximum tree depth of 10). This is 1.2% higher than the 74.4% accuracy reported in previous work.

Next, the feasibility of applying Method I and Method II to solve this problem is evaluated in the compressed domain. There are two types of features stored in the dataset: (i) steady-state, and (ii) exponential moving average. A steady-state feature is derived from the maximum and minimum values of the sensor data streams, and is thus nonlinear. Two major steps are needed to extract an exponential moving average feature: the exponential moving average transform and minimum/maximum value extraction. The first step is linear, whereas the second step is not. In the first step, the exponential moving average transform maps an incoming data stream \vec{r} to its exponential moving average series \vec{y}. An instance of \vec{y} at time t, denoted by y(t), is linearly dependent on y(t−1) and two data instances r(t) and r(t−1), which are instances of \vec{r} at time t and t−1, respectively. Hence, each y(t) can be expanded as a linear combination of r(k), k≤t, through iterative expansion of y(t′), t′<t. However, the second step, which extracts the maximum and minimum values from \vec{y}, is nonlinear. This makes it impossible to derive the exponential moving average feature through linear transformations.

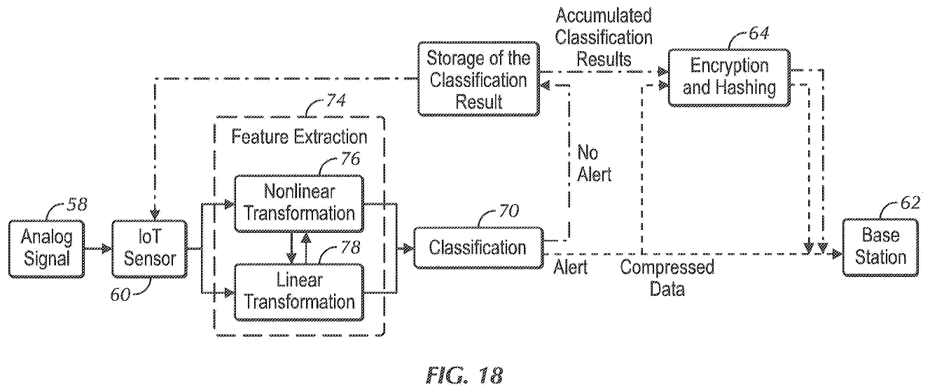

Both the steady-state and exponential moving average features are nonlinear. However, nonlinearity is only introduced due to the need to acquire the maximum and minimum values. These values can be easily captured through a series of comparisons. To handle the nonlinear features, we add a new embodiment to the disclosed architecture is shown in FIG. 18. It contains both a linear transformation block 78 and nonlinear transformation block 76 in its feature extraction stage 74. Data flow between these two blocks 76, 78 is governed by the required feature extraction for the target application. For example, in this application, the nonlinear transformation block 76 computes the maximum/minimum values of its input. It accepts raw data inputs from the IoT sensors 60 as well as the series of exponential moving averages from the linear transformation block 78. As a result, this approach enables the derivation of both the steady-state and exponential moving average features for this application. Since the data transmission protocol is BLE, the encryption and hashing energy is not modeled separately.

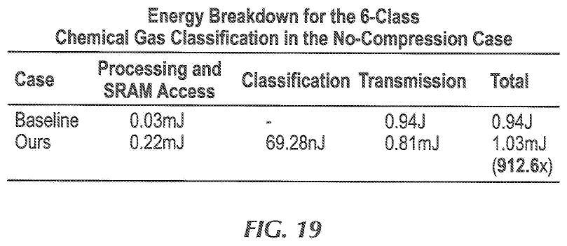

The results are summarized in the table in FIG. 19. Its columns depict the case, processing and SRAM access, classification energy, transmission energy, and total energy consumption for this application. The first row presents the energy values for conventional sense-and-transmit approach. The second row presents the energy values for the disclosed approach. In the Nyquist domain, the disclosed architecture achieves a 912.6× energy reduction relative to the baseline. Compressed-domain analysis was not conducted due to unavailability of \vec{r} and \vec{y}.

It is observed that the extraction of the maximum/minimum values incurs very little energy overhead: it only consumes 23.64 nJ extra energy on top of the 50.98 μJ of MAC and 0.17 μJ of SRAM energy needed for the linear transformation. This demonstrates the disclosed approach can also cover nonlinear features.

Conclusion

As such, disclosed herein is a novel IoT sensor architecture that is smart, secure, and energy-efficient. The IoT sensor designer can choose from among many paths through this architecture based on which one is the most suitable for the targeted IoT application. The architecture was evaluated on IoT applications picked from different domains: arrhythmia detection, Parkinson's disease freezing of gait detection, epileptic seizure detection, neural prosthesis spike sorting, human activity detection, and chemical gas detection. The classification accuracy and energy consumption of the architecture was investigated. It was shown that the classification accuracies were comparable or better than the state-of-the-art for these applications, yet with energy consumption up to three orders of magnitude lower than IoT sensors based on the traditional sense-and-transmit approach.

It is understood that the above-described embodiments are only illustrative of the application of the principles of the present invention. The present invention may be embodied in other specific forms without departing from its spirit or essential characteristics. All changes that come within the meaning and range of equivalency of the claims are to be embraced within their scope. Thus, while the present invention has been fully described above with particularity and detail in connection with what is presently deemed to be the most practical and preferred embodiment of the invention, it will be apparent to those of ordinary skill in the art that numerous modifications may be made without departing from the principles and concepts of the invention as set forth in the claims.

1-37. (canceled)

38. An Internet of Things (IoT) sensor architecture comprising:
one or more IoT sensor components configured to capture data; and
one or more processors configured to analyze the captured data, the processors comprising:
a data compression module configured to convert received data into compressed data;
a machine learning module comprising a feature extraction module configured to extract features from the received data and a classification module configured to classify the extracted features and implement one of alert notification and continuous notification based on the extracted feature classification; and
an encryption/hashing module configured to encrypt and ensure integrity of resulting data from the machine learning module or the received data.

39. The IoT sensor architecture of claim 38, wherein the IoT sensor component comprises an analog-to-digital conversion module configured to convert a received analog signal comprising captured data into a digital signal for analysis by the processors.

40. The IoT sensor architecture of claim 38, wherein the data compression module is configured to implement one of compressive sensing and CSP.

41. The IoT sensor architecture of claim 38, wherein the feature extraction module is configured to extract features in one of a compressed domain and a Nyquist domain.

42. The IoT sensor architecture of claim 38, wherein the feature extraction module comprises a linear transformation module and a nonlinear transformation module.

43. The IoT sensor architecture of claim 38, wherein the machine learning module is configured to implement one of random forest, adaptively boosted decision tree, and K-means.

44. The IoT sensor architecture of claim 38, wherein the encryption/hashing module is configured to implement advanced encryption standard (AES) for encryption and secure hash algorithm (SHA) for integrity checking.

45. The IoT sensor architecture of claim 38, wherein the resulting data is transmitted to one of a base station and a user-side application.

46. The IoT sensor architecture of claim 45, wherein the resulting data transmission is implemented via one of Bluetooth low energy (BLE), Zigbee, and medical implant communication service (MICS).

47. A method for processing captured data on an Internet of Things (IoT) sensor architecture, the method comprising:
capturing data via one or more IoT sensor components;
analyzing the captured data via one or more processors, the analysis comprising:
compressing received data via a data compression module;
extracting features from the received data via a feature extraction module;
classifying the extracted features via a classification module;
implementing one of an alert notification and continuous notification via the classification module; and
encrypting and ensuring integrity of resulting data from the machine learning module or the received data via an encryption/hashing module.

48. The method of claim 47, wherein capturing data further comprises converting an analog signal into a digital signal via an analog-to-digital conversion module.

49. The method of claim 47, wherein compressing the received data is implemented via one of compressive sensing and CSP.

50. The method of claim 47, wherein extracting features occurs in one of a compressed domain and a Nyquist domain.

51. The method of claim 47, wherein encrypting and checking integrity of resulting data further comprises implementing advanced encryption standard (AES) and secure hash algorithm (SHA), respectively.

52. The method of claim 47, further comprising transmitting the resulting data to one of a base station and a user-side application.

53. The method of claim 52, wherein transmitting the resulting data occurs via one of Bluetooth low energy (BLE), Zigbee, and medical implant communication service (MICS).

54. A non-transitory computer-readable medium having stored thereon a computer program for execution by a processor configured to perform a method for processing captured data on an Internet of Things (IoT) sensor architecture, the method comprising:
capturing data via one or more IoT sensor components;
analyzing the captured data via one or more processors, the analysis comprising:
compressing received data via a data compression module;
extracting features from the received data via a feature extraction module;
classifying the extracted features via a classification module;
implementing one of an alert notification and continuous notification via the classification module; and
encrypting and ensuring integrity of resulting data from the machine learning module or the received data via an encryption/hashing module.

55. The computer-readable medium of claim 54, wherein capturing data further comprises converting an analog signal into a digital signal via an analog-to-digital conversion module.

56. The computer-readable medium of claim 54, wherein compressing the received data is implemented via one of compressive sensing and CSP.

57. The computer-readable medium of claim 54, wherein extracting features occurs in one of a compressed domain and a Nyquist domain.

58. The computer-readable medium of claim 54, wherein encrypting and checking integrity of resulting data further comprises implementing advanced encryption standard (AES) and secure hash algorithm (SHA), respectively.

59. The computer-readable medium of claim 54, further comprising transmitting the resulting data to one of a base station and a user-side application.

60. The computer-readable medium of claim 59, wherein transmitting the resulting data occurs via one of Bluetooth low energy (BLE), Zigbee, and medical implant communication service (MICS).

61. An Internet of Things (IoT) sensor architecture comprising:
one or more IoT sensor components configured to capture data; and
one or more processors configured to analyze the captured data, the processors comprising:
a data compression module configured to convert received data into compressed data;
a machine learning module comprising a feature extraction module configured to extract features from the received data based on a linear transformation and a nonlinear transformation and a classification module configured to classify the extracted features; and
an encryption/hashing module configured to encrypt and ensure integrity of resulting data from the machine learning module or the received data.

62. The IoT sensor architecture of claim 61, wherein the IoT sensor component comprises an analog-to-digital conversion module configured to convert a received analog signal comprising captured data into a digital signal for analysis by the processors.

63. The IoT sensor architecture of claim 61, wherein the data compression module is configured to implement one of compressive sensing and CSP.

64. The IoT sensor architecture of claim 61, wherein the feature extraction module is configured to extract features in one of a compressed domain and a Nyquist domain.

65. The IoT sensor architecture of claim 61, wherein the classification module is configured to implement one of alert notification and continuous notification.

66. The IoT sensor architecture of claim 61, wherein the machine learning module is configured to implement one of random forest, adaptively boosted decision tree, and K-means.

67. The IoT sensor architecture of claim 61, wherein the encryption/hashing module is configured to implement advanced encryption standard (AES) for encryption and secure hash algorithm (SHA) for integrity checking.

68. The IoT sensor architecture of claim 61, wherein the resulting data is transmitted to one of a base station and a user-side application.

69. The IoT sensor architecture of claim 68, wherein the resulting data transmission is implemented via one of Bluetooth low energy (BLE), Zigbee, and medical implant communication service (MICS).
